(19) United States
(12) Patent Application Publication
(10) Pub. No.: US 2009/0274732 A1
(43) Pub. Date: Nov. 5, 2009

(54) Type-2 Diabetes Combination Wafer

(75) Inventors: Hans-Rainer Hoffmann, Neuwied (DE); Reto Brandli, San Francisco, CA (US); Frank Theobald, Wehr (DE)

Correspondence Address: D. PETER HOCHBERG CO. L.P.A., 1940 EAST 6TH STREET, CLEVELAND, OH, 44114, US

(73) Assignee: LTS LOHMANN THERAPIE-SYSTEME AG, Andernach (DE)

(21) Appl. No.: 12/308,242

(22) PCT Filed: Jun. 4, 2007
(86) PCT No.: PCT/EP2007/004953

§ 371 Date: Dec. 10, 2008

(30) Foreign Application Priority Data
Jun. 16, 2006 (DE) 10 2006 027 790.2

(51) Int. Cl. A61K 9/00; A61K 31/165; A61P 3/10

(52) U.S. Cl. 424/400; 514/617

(57) ABSTRACT

Rapidly disintegrating oral dosage forms for the application of active agent combinations for diabetes therapy. The dosage forms contain at least two active agents suitable for treating type-2 diabetes. The antidiabetic active agents are selected from the group comprising sulfonylureas, glitazones, glinides, biguanides, and absorption-delaying agents. The use of the active agent combination to produce an oral dosage form for the treatment of diabetes, a method for the therapeutic treatment of diabetes, and a method for the production of a sheet-like dosage form are also disclosed.

### CROSS-REFERENCE TO RELATED APPLICATIONS

This application is a National Stage application of International Application No. PCT/EP2007/004953, filed on Jun. 4, 2007, which claims priority of German application number 10 2006 027 790.2, filed on Jun. 16, 2006, both of which are incorporated herein by reference in their entireties.

### BACKGROUND OF THE INVENTION

1. Field of the Invention

The present invention relates to rapidly disintegrating oral dosage forms for the application of active agent combinations for the therapy of diabetes.

2. Description of the Prior Art

Diabetes mellitus is a metabolic disease that affects around six million people in Germany alone. Diabetes mellitus, commonly also termed diabetes, is considered to be a long-lasting pathological elevation of the blood-sugar level caused by the body's insufficient ability to utilise carbohydrates.

With diabetes mellitus, a distinction is made between Type 1 and Type 2 diabetes. Both of these manifestations can occur independent of age.

Type 1 diabetes, also referred to as “juvenile” or “insulin-dependent” diabetes, as a rule occurs already in children and adolescents, but may also manifest itself at a later age, in adults. Type 1 diabetes is present in about 10% of diabetics and occurs as a result of the destruction of insulin-producing cells by the immune system (autoimmune disease).

Since in type 1 diabetics the body is no longer able to produce insulin, type 1 diabetes always requires treatment with insulin and therefore does not react positively to oral therapy.

Type 2 diabetes, also termed “diabetes of old age” or “insulin-independent diabetes” and accounting for 90% of diabetes cases, develops only slowly and as a rule appears only in elderly people. However, due to changing lifestyle and eating habits, it is also, increasingly, diagnosed in overweight children and adolescents.

A precursor of type 2 diabetes is the so-called pathological glucose tolerance, wherein the body is no longer able to utilise carbohydrates properly and which is often concurrent with overweight, high blood pressure, high blood lipid levels and elevated uric acid levels, summarized under the term “metabolic syndrome”.

One of the main causes of type 2 diabetes is insulin resistance, i.e. loss of action of insulin, occurring as a result of overnutrition and reduced physical activity.

Experts anticipate that in the coming years the number of type 2 diabetics is going to increase still further since people become more overweight and grow older and a generation of overweight adolescents reaches adulthood. The onset of type 2 diabetes is insidious and is often diagnosed only very late. Due to the continuous progressive development of the disease, treatment requires regular control and may require adaptation to the course of the disease.

The aim of the treatment is to maintain blood glucose at the level of a non-diabetic since a chronic elevated blood glucose level may lead to serious damage to the vascular and nervous system, as well as damage the heart, eyes and kidneys.

As in type 2 diabetics, at the beginning of the disease there is no absolute insulin deficiency but merely a diminished action of that hormone, there are different approaches to oral therapy, involving different active agents and actions of mechanism, which are based on the following:

improving sensitivity of body cells to insulin;

improving insulin secretion in the pancreas;

stimulation of endogenous insulin secretion specifically during meals to avoid postprandial hyperglycaemia;

delaying glucose absorption and delaying the degradation of carbohydrates.

Once a type 2 diabetes has reached a state requiring treatment, medicaments to treat type 2 diabetes will have to be taken for life.

In the treatment of type 2 diabetes it may be found expedient and therapeutically indicated to combine two oral antidiabetics in order to achieve a better therapeutic effect or to achieve a reduction of the dose and thereby of the side effect profile for a class of substances.

However, the combination of active agents also requires consistent intake in accordance with an intake schedule in order to achieve the desired therapeutic effect. For this reason it is desirable to combine the active agents in one dosage form as this facilitates intake for the patient and minimises the risk of false application.

Easy and direct application of the dosage forms should therefore be possible, in order to facilitate intake for the patient and increase compliance.

The dosage form should furthermore be capable of releasing the active agents quickly and ensuring a quick onset of action. The disintegration of the dosage form and the release of the active agents should therefore take place already at the site of application, in the case of dosage forms for oral application, for example, already in the oral cavity. This is of particular importance when taking certain active agents immediately prior to ingestion of food or for treating hyperglycaemic shock.

### SUMMARY OF THE PRESENT INVENTION

The object of the present invention was therefore to provide a dosage form that can be effectively used in the treatment of diabetes and which requires only a low dosage of active agents in order to keep the side effects of the antidiabetics as low as possible and thereby not to interfere with daily life. In addition, the dosage form should exhibit good compliance, which means that administration to the patient should be as simple as possible and the patient should have no reservations against taking the medication, for instance on account of the size of the dosage form, or the like. The dosage form is furthermore intended to avoid the disadvantages of known dosage forms, particularly tablets.

As explained above, in order to maintain a constant blood glucose level it is indispensable for diabetics to take their medications regularly. Moreover, it must be possible to apply the medicaments in a simple manner so that, if necessary, they can be taken prior to meals, as well as in public, since with certain active agents from the group of antidiabetics it is helpful if they are applied before meals.

In the case of pathoglycaemias, too, application must occur quickly to counter hyperglycaemic shock.

Dosage forms commonly utilised for administration of active agents to treat diabetes are tablets and capsules.

Tablets or capsules are relatively easy to take, but onset of action is generally delayed and the active agents, on being absorbed via the gastrointestinal tract, are subject to the “first-pass effect”, thus necessitating high initial active agent concentrations in the tablet or capsule.

Often, patients are only able to take tablets with a liquid, which leads to a certain reduction in their freedom of movement. Only with difficulty, for instance, is it possible to take a tablet while driving a car or during a meeting immediately prior to a meal.

### DETAILED DESCRIPTION OF THE PRESENT INVENTION

It was found that the above object is solved by means of sheet-like dosage forms of a hydrophile polymer film which disintegrates in the oral cavity, the dosage forms containing at least two active agents which are suitable for the treatment of type 2 diabetes.

In addition to the known active agents, their free acids or bases, or their therapeutically active salts, are also suitable as active agents.

By combining the active agents in the dosage form according to the invention, it is easier for the patient to take both of the active agents. Absorption of the active agents via the oral mucosa, as compared to other peroral dosage forms, affords, for instance, the advantages that patients also having difficulty swallowing or patients refusing to take tablets can be administered medicaments via the oral route. In addition, the risk of medication errors is reduced as the patient has to take only one medicament for both active agents. This improves compliance and therapy success.

In addition, since the active agents or at least parts of active agents can be absorbed directly via the mucous membrane, the time until the onset of action occurs can be markedly reduced, so that glinides or absorption-delaying agents can be applied in accordance with the requirements of the application.

Especially due to the combining of active agents in one administration form for treating diabetes, wherein one of the active agents is a quick-acting active agent, such as a glinide, or an absorption retardant, and the second one is an active agent having, a longer half-life for long-term increase of the insulin secretion, it is possible to achieve special advantages. For instance, if such a combination is taken before meals, the blood-sugar level can be regulated such that it does not leave the normal range, even immediately while a meal is being taken and shortly thereafter, and a postprandial hyperglycaemia is avoided.

Furthermore, a single active agent combination may contain active agents with different mechanisms of action having a synergistic effect, so that as a result of the different physiological activity lower amounts of active agents can be dosed in blood-sugar checks than would be the case with single-component compositions.

Even where active agents are combined, a consistent intake and good compliance of the medicament are a prerequisite to ensuring optimal efficacy.

The administration of these active agent combinations in sheet-like dosage forms (wafers) not only enables easy intake but also the exact adaptation of the active agent components to each other, so that false dosages because intake has been forgotten or because of double intake of only one active agent, and consequently an insufficient regulation of the blood-sugar level, which may trigger hyper- or hypoglycaemic states, do not occur.

Another advantage of the transmucosal administration of certain active agents is the fact that the gastrointestinal route is circumvented and the “first pass” effect following peroral administration, that is, the metabolism of a significant portion of the active agent during the first liver passage, is thereby avoided, so that the active agent is utilised to a high degree.

In addition, as loss of active agent caused by the first-pass effect does practically not occur, the dosage of certain active agents can often be lowered correspondingly, which likewise leads to the patient being disburdened, and to improved well-being as a consequence of lower UDE's.

By varying the ratio of the active agents to each other, it is, in addition, possible to adapt the dosages to the respective needs. Thus, the dosage forms according to the invention may contain active agent combinations that are adapted to the patient, depending on whether the patient responds better to carbohydrate absorption inhibitors or to raising insulin secretion.

Because of the simple and low-cost manufacture of the wafers, it is possible to provide a large number of medicaments containing different active agent concentrations.

If the wafer is made up of a laminate, it is possible, for example, to alter only the layer thickness of an active agent-containing layer, or to alter the concentration of the active agent.

On the other hand, pharmaceutical products can be produced which have different active agent contents but the same active agent ratio, simply by means of cutting the surface of the dosage form to different sizes.

Furthermore, because of their flat shape the wafers of the invention, which contain the active agent combinations, can be carried along easily, for example in a wallet, and are available at once, even when travelling. They are easy to take and they take effect quickly, both in the treatment of diabetes and in cases of suddenly occurring hypoglycaemia.

Water-soluble or swellable polymers that are suitable as a base polymer for the hydrophilic water-soluble and/or swellable polymer film are polymers from the group comprising dextran, polysaccharides, inclusive of starch and starch derivatives, cellulose derivatives, such as carboxymethyl cellulose, ethyl cellulose or propyl cellulose, hydroxypropylmethyl cellulose, hydroxypropyl cellulose, sodium carboxymethyl cellulose (e.g. WALOCEL®), methyl cellulose, hydroxyethyl cellulose and hydroxypropylethyl cellulose, polyvinyl alcohols, polyethylene glycols, polyacrylic acids, polyacrylates, polyvinylpyrrolidones, alginates, pectins, gelatine, alginic acid, collagen, chitosan, arabinogalactan, galactomannan, agar-agar, agarose, carrageenan natural gums, tragacanth, highly dispersed silicon dioxide, bentonite, as well as derivatives of the aforementioned hydrophilic polymers or combinations of two or more of these polymers. As an alternative, the polymer film may be made of a polyvinyl alcohol-polyethylene glycol graft copolymer.

The proportion of polymer contained in a dosage form according to the invention is preferably 5 to 95%-wt., more preferably 15 to 75%-wt., relative to the dry mass of the dosage form.

The inventive sheet-like pharmaceutical preparations which are based on hydrophilic polymers and which are used for the treatment of type 2 diabetes contain an active agent combination of at least two active agents which are suitable for oral therapy of type 2 diabetes.

In a preferred embodiment, the pharmaceutical preparation contains two to four, preferably two to three, and more preferably two, active agents, with the active agents being selected from the group which comprises the sulfonylureas, glitazones, glinides, biguanides and absorption-delaying agents.

Preferably, the active agents contained in the active agent preparation belong to different active agent classes with different mechanisms of action.

It is furthermore preferred that one of the active agents is an active agent which quickly lowers the blood-sugar level while the second active agent develops a long-term action.

A preferred embodiment of the pharmaceutical preparation contains an active agent combination of two active agents wherein said active agents are selected from the group comprising pioglitazone, rosiglitazone, nateglinides, repaglinides, glibenclamide, glibornuride, glimepiride, gliquidone and tolbutamide.

Another combination of active agents contains nateglinides and metformin.

The active agent content of the antidiabetics is between 2% and 80%, preferably between 5% and 70% and more preferably between 10% and 30%, relative to the total weight of the wafer.

To improve the physicochemical properties, for example reduce the brittleness or embrittlement, humectants, such as glycerine, propylene glycol, sorbitol, mannitol, polyethylene glycol, polyglycerol ester and the like, may be added to the film.

In a further embodiment, antioxidants, for example vitamin C (ascorbic acid), ascorbyl palmitate, vitamin E (tocopherol acetate), hydroxybenzoic acid derivatives, may be added to the wafer, in order to stabilise the film and the active agents. Furthermore, acidic and basic ion exchangers may be used as stabilisers.

In further embodiments, further ingredients such as dyes, pigments, taste flavourings, natural and/or synthetic flavouring substances, sweeteners, buffering systems, may be added to the film. In particular, the taste flavourings and flavouring substances can cover the often bad inherent taste or smell of the active agents and/or give the dosage form a pleasant taste, so that the patient's readiness to take the medication is considerably improved.

The addition of buffering systems on the one hand serves to stabilise the film and the active agents against outside influences and during storage. On the other hand, the pH of the dosage form can thereby be adjusted to a physiologically acceptable pH value so that irritation of mucous membranes is avoided. By using a buffering system, it is also possible to improve the solubility of acidic or basic active agents in the matrix.

The dosage forms according to the invention are configured so as to be thin, for example in the form of a wafer. The thickness of the dosage form is preferably 0.1 to 5 mm, more preferably 0.5 to 1 mm. The lower limit for the thickness of the dosage form is about 50 μm. The surface area of the dosage form is between 0.09 cm² and 12 cm², preferably between 1 cm² and 8 cm², and more preferably between 3 cm² and 6 cm².

In a further embodiment, the wafers of the present invention contain a disintegrant or a wicking agent, for example a bicarbonate-acid mixture or an aerosil, being activated by contact with a liquid and accelerating the disintegration of the wafer after application thereof, and thereby also accelerating the release of active agent

In a preferred embodiment, the wafer is present as a foam so that the release of active agent takes place even more rapidly because of the enlarged surface. In this embodiment, the cavities of the foam may contain one or more of the active agents in liquid form.

To improve the absorption of the active agents via the mucous membrane, permeation enhancers, such as substances from the groups of the fatty alcohols, fatty acids, polyoxyethylene fatty alcohol ethers, polyoxyethylene fatty acid esters, fatty alcohol esters and fatty acid esters, particularly sorbitan monolaurate or esters of long-chain fatty acids with methyl, ethyl or isopropyl alcohol, or esters of fatty alcohols with acetic acid or lactic acid, or substances such as DMSO (dimethyl sulfoxide) and oleic acid diethanolamine may likewise be incorporated in the film. The constituent amount of these substances is 0.1 to 25%-wt., preferably 1 to 10%-wt., in each case relative to the total weight of the active agent matrix.

Furthermore, the composition of the wafer may contain compounds that retard the release of active agent (e.g., microencapsulation).

In a further embodiment, the wafer has mucoadhesive properties, so that it adheres to the mucous membrane until it is completely dissolved.

In a preferred embodiment, at least one of the active agents is bound to an ion exchanger, so that the hydrophilic polymer disintegrates quickly in the oral cavity, whereas the release of active agent is retarded or occurs when the pH has changed, e.g. in the gastrointestinal tract. In this way, active agents having a different mechanism of action and absorption can be administered in one dosage form, that is, at least one of the released active agents is either absorbed at the site of application, for example via the mucous membrane, or it is transported farther and absorbed at another site.

The wafer may also be made up as a laminate with different layers, with the active agents being contained in discrete layers which are spatially separated from each other and differ from each other in terms of their composition. In this way, the active agents can be released at different sites of action, but also with retardation, if the disintegration times of the various layers of the wafer differ from each other.

Likewise, the active agents may be arranged within layers that disintegrate at different rates, so that the preparation as a whole shows a retardation effect.

In a further embodiment, one of the outer layers may be mucoadhesive to promote the adherence of the dosage form on the mucous membrane and to facilitate the active agent absorption via the mucous membrane by establishing direct contact.

The disintegration of the inventive dosage form in an aqueous medium preferably takes place in the range from 1 second to 5 minutes, more preferably in a range from 5 seconds to 1 minute, and most preferably in the range from 10 seconds to 30 seconds.

The dosage forms according to the invention are advantageously suitable for administering medicaments in the oral cavity or for rectal, vaginal or intranasal administration. They can be used in human medicine as well as in veterinary medicine.

The present invention furthermore relates to the use of an active agent combination according to the invention for the production of an oral dosage form for treating diabetes, said dosage form preferably being formulated as a wafer.

Furthermore, the present invention relates to a method for the therapeutic treatment of a person suffering from diabetes, wherein the administration of an above-described active agent combination of antidiabetics is carried out by means of an orally applicable dosage form with transmucosal absorption.

Finally, the present invention also relates to a method for the production of a sheet-like dosage form, comprising the following steps:

preparing a solution containing at least one polymer and at least two antidiabetic active agents;

spread-coating the solution on a coating substrate, and

solidifying the spread-coated solution by drying and withdrawing the solvent.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. A sheet-like pharmaceutical preparation based on hydrophilic polymers, which rapidly disintegrates upon contact with moisture and which is used to treat type-2 diabetes, said pharmaceutical preparation comprising an active agent combination of at least two active agents which are suitable for the oral treatment of type-2 diabetes.

2. The pharmaceutical preparation according to claim 1, wherein the active agents are selected from the group consisting of sulfonylureas, glitazones, glinides, biguanides and absorption-delaying agents.

3. The pharmaceutical preparation according to claim 1, wherein the active agent combination contains two active agents, with said active agents being selected from the group consisting of pioglitazone, rosiglitazone, nateglinides, repaglinides, glibenclamide, glibornuride, glimepiride, gliquidone and tolbutamide.

4. The pharmaceutical preparation according to claim 3, wherein the active agent combination contains nateglinide and metformin.

5. The pharmaceutical preparation according to claim 1, wherein the hydrophilic polymer is selected from the group consisting of dextran, polysaccharides, inclusive of starch and starch derivatives, cellulose derivatives, polyvinyl alcohols, polyethylene glycols, polyacrylic acids, polyacrylates, polyvinylpyrrolidones, alginates, pectins, gelatine, alginic acid, collagen, chitosan, arabinogalactan, galactomannan, agar-agar, agarose, carrageenan natural gums, tragacanth, highly dispersed silicon dioxide, bentonite, as well as derivatives of the aforementioned hydrophilic polymers or combinations of two or more of said polymers.

6. The pharmaceutical preparation according to claim 1 in the form of a polymer film, wherein the polymer film is made of a polyvinyl alcohol-polyethylene glycol graft copolymer.

7. The pharmaceutical preparation according to claim 1, wherein said preparation further comprises a humectant selected from the group consisting of glycerine, propylene glycol, sorbitol, mannitol, polyethylene glycol and polyglycerol ester.

8. The pharmaceutical preparation according to claim 1, wherein the preparation further comprises an antioxidant selected from the group consisting of vitamin C (ascorbic acid), ascorbyl palmitate, vitamin E (tocopherol acetate) and hydroxybenzoic acid derivatives.

9. The pharmaceutical preparation according to claim 1, wherein the active agent of the preparation is bound to an acidic or basic ion exchanger for taste masking.

10. The pharmaceutical preparation according to claim 1, wherein the preparation further comprises dyes and/or pigments.

11. The pharmaceutical preparation according to claim 1, wherein the preparation further comprises natural and/or synthetic flavouring substances.

12. The pharmaceutical preparation according to claim 1, wherein the preparation further comprises a disintegrant or a wicking agent.

13. The pharmaceutical preparation according to claim 1, further comprising a buffer system for adjusting the pH value of the preparation.

14. The pharmaceutical preparation according to claim 1, wherein the hydrophilic polymer disintegrates within less than 5 minutes after application in the oral cavity of a user.

15. The pharmaceutical preparation according to claim 1, wherein the hydrophilic polymer disintegrates quickly in the oral cavity of a user whereas the active agent remains bound to an ion exchanger which releases said active agent only upon reaching the gastrointestinal tract.

16. The pharmaceutical preparation according to claim 1, wherein the active agents are contained in discrete layers which are spatially separated from each other and which differ from each other in terms of the respective composition.

17. The pharmaceutical preparation according to claim 1, wherein the preparation is present as a foam having cavities and at least one of the active agents is present in liquid form within the cavities of said foam.

18. Use of a pharmaceutical preparation according to claim 1 for rectal, vaginal or intranasal administration of pharmaceutical active agents to humans or animals.

19. Use of an active agent combination of at least two oral antidiabetic active agents for the production of an oral dosage form according to claim 1 for treating pain disorders.

20. The use according to claim 19, wherein the pharmaceutical product is formulated as a wafer.

21. A method for the therapeutic diabetes treatment of a person suffering from type-2 diabetes, comprising the step of administering an active agent combination of two antidiabetics by an orally applicable dosage form with transmucosal absorption.

22. A method for producing a sheet-like dosage form comprising hydrophilic polymers which rapidly disintegrates upon contact with moisture and which is used to treat type-2 diabetes, said pharmaceutical preparation comprising an active agent combination of at least two active agents which are suitable for the oral treatment of type-2 diabetes, said method comprising the steps of:
preparing a solution containing at least one polymer and at least two active agents;
spread-coating the solution on a coating substrate; and
solidifying the spread-coated solution by drying and withdrawing the solvent.

23. The pharmaceutical preparation according to claim 5, wherein said cellulose derivatives are selected from the group consisting of carboxymethyl cellulose, ethyl cellulose or propyl cellulose, hydroxypropylmethyl cellulose, hydroxypropyl cellulose, sodium carboxymethyl cellulose, methyl cellulose, hydroxyethyl cellulose and hydroxypropylethyl cellulose.

24. The pharmaceutical preparation according to claim 14, wherein the hydrophilic polymer disintegrates within less than 3 minutes after application in the oral cavity.

25. The pharmaceutical preparation according to claim 24, wherein the hydrophilic polymer disintegrates within less than 1 minute after application in the oral cavity.

26. The pharmaceutical preparation according to claim 25, wherein the hydrophilic polymer disintegrates within less than 30 seconds after application in the oral cavity.
